(19) United States
(12) Patent Application Publication
(10) Pub. No.: US 2024/0194336 A1
(43) Pub. Date: Jun. 13, 2024

(54) SYSTEMS, METHODS, AND APPARATUSES FOR SECURE DIAGNOSIS AND TREATMENTS AND AUTHENTICATING DIAGNOSED USERS

(71) Applicant: Robb Takeshi Fujioka, Manhattan Beach, CA (US)

(72) Inventor: Robb Takeshi Fujioka, Manhattan Beach, CA (US)

(21) Appl. No.: 18/385,530

(22) Filed: Oct. 31, 2023

Related U.S. Application Data
(63) Continuation of application No. 17/352,159, filed on Jun. 18, 2021, now abandoned.
(60) Provisional application No. 63/041,080, filed on Jun. 18, 2020.

(51) Int. Cl. G16H 40/67; A61B 5/00; A61B 5/0205; A61B 5/021; A61B 5/024; A61B 5/08; A61B 5/145; G06Q 10/105; G07C 9/00; G16H 10/60; G16H 40/20; G16H 50/20; H04W 4/029; H04W 4/80

(52) U.S. Cl. CPC G16H 40/67; A61B 5/002; A61B 5/02055; A61B 5/7425; G06Q 10/105; G16H 10/60; G16H 40/20; G16H 50/20; A61B 5/021; A61B 5/024; A61B 5/08; A61B 5/14542; A61B 2503/20; G07C 9/00; H04W 4/029; H04W 4/80

(57) ABSTRACT

Disclosed herein are systems and methods for monitoring a patient. One of the systems for monitoring a patient includes: a plurality of health sensors configured to measure one or more vital signs of a patient; and a health application residing on the mobile device, the health application is configured to communicate with the plurality of health sensors and to receive data of the one or more vital signs measured by the plurality of health sensors.

### CROSS-REFERENCE TO RELATED APPLICATIONS

The present application is a continuation of U.S. patent application Ser. No. 17/352,159, filed Jun. 18, 2021, which claims priority under to U.S. Provisional Patent Application No. 63/041,080, filed Jun. 18, 2020, the disclosures of which are hereby incorporated by reference in its entireties. This application is also related to PCT Patent Application No. PCT/US20/25840, filed Mar. 30, 2020, the disclosure of which is also hereby incorporated by reference in its entirety for all purposes.

### FIELD

The subject matter described herein relates generally to systems, methods, apparatuses, and techniques for securely diagnosing and providing treatments and for authenticating the diagnosed users.

### BACKGROUND

The COVID-19 pandemic has caused massive disruptions to employment as governments mandated stay-at-home and business closures. However, businesses will eventually be allowed to reopen, and workers will need to return to work. To this end, the US Centers for Disease Control and Prevention (CDC) has issued a guidance for reopening. The guidance includes, among others: 1. PRACTICE GOOD HYGIENE & IMPLEMENT PERSONAL PROTECTIVE MEASURES: Hand washing, cough etiquette and face covering (education). 2. SOCIAL DISTANCING: Maintaining physical distance between persons (6 feet). 3. ENVIRONMENTAL SURFACE CLEANING: Proper cleaning and disinfecting of surfaces (education). 4. MONITOR FOR INDICATIVE SYMPTOMS—DAILY ROUTINE HEALTH CHECKS: Daily, routine health checks at home and workplace (under the supervision of health professional—Registered Nurse, Doctor). 5. CONTACT TRACING FOLLOWING COVID+TEST: COVID testing—at home antibody testing (under supervision of Doctor), PCR testing—via nationwide certified laboratory.

However, there are currently no effective systems, apparatuses, methods and processes for employees and employers to diagnose and monitor current health checks/statuses of the employees to allow and verify the employees to safely return to work, and if not to provide for the employees to see a health professional.

Thus, needs exist for systems, apparatuses, computer program products, methods, processes and techniques associated with diagnosing, monitoring current health checks/statuses of the employees, providing for the employees to see a health professional, and authenticating the employees at a location, without the above mentioned and other disadvantages.

### SUMMARY

Provided herein are example embodiments of systems, apparatuses, computer program products, techniques, methods and processes associated with diagnosing, monitoring current health checks/statuses of patients (e.g., employees), providing for the patients to see a health professional, and authenticating a person, for example, at a work location or an event.

One of the systems includes: a plurality of health sensors configured to measure one or more vital signs of a patient; and a health application residing on the mobile device, the health application is configured to communicate with the plurality of health sensors and to receive data of the one or more vital signs measured by the plurality of health sensors.

The plurality of health sensors can be one or more of blood oxygen level monitor, a blood pressure monitor, a thermometer, a hear rate monitor, and a breathing monitor. Each of the plurality of health sensors is configured to communicate with the health application via Bluetooth. The health application is also configured to display data of the one or more vital signs measured by the plurality of health sensors.

In some embodiments, the health application is configured to determine a location of the patient; and determine nearby pharmacies based on the location of the patient. The health application can also evaluate the data of the one or more vital signs to provide work clearance or to recommend further treatments.

The systems, methods, processes and apparatuses for diagnosing, monitoring current health checks/statuses of the employees, providing for the employees to see a health professional, and authenticating the employees described herein in detail are only example embodiments and should not be considered limiting. Other configurations, methods, features and advantages of the subject matter described herein will be or will become apparent to one with skill in the art upon examination of the following figures and detailed description. It is intended that all such additional configurations, methods, features and advantages be included within this description, be within the scope of the subject matter described herein, and be protected by the accompanying claims. In no way should the features of the example embodiments be construed as limiting the appended claims, absent express recitation of those features in the claims.

Generally, the present disclosure provides embodiments of systems, apparatuses, computer program products, techniques, methods and processes associated with diagnosing, monitoring current health checks/statuses of the employees, providing for the employees to see a health professional, and authenticating the employees, for example, at a work location or an event. In some embodiments, these systems, methods, apparatuses, computer program products, and techniques can be configured to provide a scalable, easy-to-use, easy-to-implement, low cost routine, daily health check. The present disclosure can include routine, daily health checks at home. The checks can include vitals such as, pulse oxygen levels, temperature & blood pressure, etc. It can also include live doctor escalation (telemedicine) if and when needed. When a user is cleared, on-premise pre-check before entry (e.g., using QR-code & infrared) can be performed. All features and functions can be HIPPA compliant.

Other features and advantages of the present invention are or will become apparent to one skilled in the art upon examination of the following figures and detailed description, which illustrate, by way of examples, the principles of the present invention.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a chart comparing the episodic and population health models.

FIG. 2 is a chart illustrating the health monitoring platform in accordance with some embodiments of the present disclosure.

FIGS. 3, 4A, and 4B are flow diagrams of data handling processes that HIPPA compliance in accordance with some embodiments of the present disclosure.

FIG. 5 is a flow diagram of a clearance process in accordance with some embodiments of the present disclosure.

FIGS. 6A and 6B are diagrams showing various applications of the health monitoring platform in accordance with an aspect of the disclosure.

FIG. 7 is a flow diagram of a clearance process in accordance with some embodiments of the present disclosure.

FIG. 8 illustrates a screenshot of a health application with display of data from a smart pulse oximeter in accordance with some embodiments of the present disclosure.

FIGS. 9-11 illustrate various features and specifications of the health application and/or the pulse oximeter in accordance with some embodiments of the present disclosure.

FIG. 12 illustrates an example smart thermometer as implemented by the health monitoring platform in accordance with some embodiments of the present disclosure.

FIG. 13 illustrates a screenshot of a health application with display of data from the thermometer in accordance with some embodiments of the present disclosure.

FIGS. 14 and 15 illustrate various features and specifications of the health application and/or the smart thermometer in accordance with some embodiments of the present disclosure.

FIG. 16 illustrates an example smart blood pressuring measuring device as implemented by the health monitoring platform in accordance with some embodiments of the present disclosure.

FIGS. 17, 18, and 19 illustrate various features, specifications, and screenshot of the health application and/or the blood pressure measuring device in accordance with some embodiments of the present disclosure.

FIG. 20 illustrates an example thermal measuring device as implemented by the health monitoring platform in accordance with some embodiments of the present disclosure.

FIGS. 21-24 illustrate various features and specifications of the health application and/or the thermal measuring device in accordance with some embodiments of the present disclosure.

FIG. 25 illustrates a hardware implementation of the health monitoring platform in accordance with some embodiments of the present disclosure.

The figures and the following description describe certain embodiments by way of illustration only. One skilled in the art will readily recognize from the following description that alternative embodiments of the structures and methods illustrated herein can be employed without departing from the principles described herein. Reference will now be made in detail to several embodiments, examples of which are illustrated in the accompanying figures. It is noted that wherever practicable similar or like reference numbers can be used in the figures to indicate similar or like functionality.

### DETAILED DESCRIPTION

### Overview

On a high level, the present disclosure relates to a patient monitoring platform that combines vital-sign sensors (e.g., health sensor), wireless connectivity, and telemedicine capability into a single platform.

This can require the integration of at least 6 technologies:
1. Vital sensors (e.g., health sensors) and devices (thermometer, blood pressure monitor, pulse oximeter)
2. Communications stack (low energy Bluetooth or NFC)
3. Telehealth services
4. Electronic Health/Medical Records (EHR/EMR)
5. Smart phone and watch data (fitness trackers and OURA ring)
6. Machine learning and Artificial Intelligence

### Vital Sensors

Vital signs (vitals) are a group of the several important medical signs that indicate the status of the body's vital (life-sustaining) functions. These measurements are taken to help assess the general physical health of a person, give clues to possible diseases, and show progress toward recovery. The normal ranges for a person's vital signs vary with age, weight, gender, and overall health.

In some embodiments, the disclosed patient monitoring platform (hereinafter “the patient monitoring platform”) can monitor four primary vital signs: body temperature, blood pressure, pulse (heart rate) and breathing rate (respiratory rate), often noted as BT, BP, HR and RR. However, depending on the clinical setting, patient monitoring platform can include other vital signs measurements called the “fifth vital sign” or “sixth vital sign”. The “fifth vital sign” can include: pain, menstrual cycle, oxygen saturation and blood glucose level. The “sixth vital sign” can include: CO2, gait speed and delirium.

Today, vital signs are recorded using the logical observation identifiers names and codes (LOINC) standard, which is an internationally accepted standard coding system. LOINC applies universal code names and identifiers to medical terminology related to electronic health records. The purpose is to assist in the electronic exchange and gathering of clinical results (such as laboratory tests, clinical observations, outcomes management and research). LOINC has two main parts: laboratory LOINC and clinical LOINC. Clinical LOINC contains a subdomain of Document Ontology which captures types of clinical reports and documents.

Body Temperature (BT): Temperature recording gives an indication of core body temperature which is normally tightly controlled (thermoregulation) as it affects the rate of chemical reactions. Body temperature is maintained through a balance of the heat produced by the body and the heat lost from the body. Body temperature is an indicator of inflammation and can be elevated due to infection or an autoimmune response. The main reason for checking body temperature is to solicit any signs of systemic infection or inflammation in the presence of a fever. Fever is considered temperature of 37.8° C. (100.04° F.) or above. Other causes of elevated temperature include hyperthermia, which results from unregulated heat generation or abnormalities in the body's heat exchange mechanisms. Temperature depression (hypothermia) also needs to be evaluated. Hypothermia is classified as temperature below 35° ° C. (95° F.). It is also recommended to review the trend of the patient's temperature over time. A fever of 38° C. does not necessarily indicate an ominous sign if the patient's previous temperature has been higher.

Heart Rate (HR) is the rate at which the heart beats while pumping blood through the arteries, recorded as beats per minute (bpm). It can also be called “heart rate”. In addition to providing the heart rate, the pulse should also be evaluated for strength and obvious rhythm abnormalities. The pulse can vary due to exercise, fitness level, disease, emotions, and medications. The pulse also varies with age. A newborn can have a heart rate of 100-160 bpm, an infant (0-5 months old) a heart rate of 90-150 bpm, and a toddler (6-12 months old) a heart rate of 80-140 bpm. A child aged 1-3 years old can have a heart rate of 80-130 bpm, a child aged 3-5 years old a heart rate of 80-120 bpm, an older child (age of 6-10) a heart rate of 70-110 bpm, and an adolescent (age 11-14) a heart rate of 60-105 bpm. An adult (age 15+) can have a heart rate of 60-100 bpm.

Respiratory Rate (RR) is the rate at which breathing occurs. This is usually measured in breaths per minute and is set and controlled by the respiratory center. Average respiratory rates vary between ages, but the normal reference range for people age 18 to 65 is 16-20 breaths per minute. Respiration rates can increase with fever, illness, or other medical conditions. The value of respiratory rate as an indicator of potential respiratory dysfunction. Respiratory rate is a clear indicator of acidotic states, as the main function of respiration is removal of CO2 leaving bicarbonate base in circulation.

Blood Pressure (BP) is the pressure of circulating blood on the walls of blood vessels. Most of this pressure is due to work done by the heart by pumping blood through the circulatory system. Used without further specification, “blood pressure” usually refers to the pressure in large arteries of the systemic circulation. Blood pressure is usually expressed in terms of the systolic pressure (maximum during one heartbeat) over diastolic pressure (minimum in between two heartbeats) and is measured in millimeters of mercury (mmHg), above the surrounding atmospheric pressure. Blood pressure measures the elasticity of blood vessels and allows us to identify risks for developing cardiovascular conditions.

Oxygen Saturation is the fraction of oxygen-saturated hemoglobin relative to total hemoglobin (unsaturated+saturated) in the blood. The human body requires and regulates a very precise and specific balance of oxygen in the blood. Normal arterial blood oxygen saturation levels in humans are 95-100 percent. If the level is below 90 percent, it is considered low and called hypoxemia. Arterial blood oxygen levels below 80 percent can compromise organ function, such as the brain and heart, and should be promptly addressed. Continued low oxygen levels can lead to respiratory or cardiac arrest. Oxygen therapy can be used to assist in raising blood oxygen levels. Oxygenation occurs when oxygen molecules (O2) enter the tissues of the body. For example, blood is oxygenated in the lungs, where oxygen molecules travel from the air and into the blood. Oxygenation is commonly used to refer to medical oxygen saturation.

In some embodiments, the patient monitoring platform can generate an early warning score (EWS), which can be to quickly determine the degree of illness of a patient. The EWS can be based on one or more of the vital signs (e.g., respiratory rate, oxygen saturation, temperature, blood pressure, pulse/heart rate, AVPU response). Scores were developed in the late 1990s when studies showed that in-hospital deterioration and cardiac arrest was often preceded by a period of increasing abnormalities in the vital signs.

The ability to intermittently or continuously measure a person vital signs is important due to the advent of novel infectious diseases such as the coronavirus disease 2019 (COVID-19), which is an infectious disease caused by severe acute respiratory syndrome coronavirus 2 (SARS-CoV-2). Common symptoms include fever, cough, fatigue, shortness of breath, and loss of smell and taste. While most cases result in mild symptoms, some progress to acute respiratory distress syndrome (ARDS) likely precipitated by a cytokine storm, multi-organ failure, septic shock, and blood clots. The time from exposure to onset of symptoms is typically around five days but can range from two to fourteen days.

COVID-19 presents a myriad of symptoms and clinicians need access to medical devices that allow them to monitor and manage those symptoms in real-time in order to create the most appropriate treatment plans for each individual. While scientists first believed the COVID-19 virus was one that attacked only the respiratory system, they have come to find out that the new coronavirus is not a one-note disease. COVID-19, researchers have found, also attacks the heart, the kidneys and the nervous system. In children, doctors are seeing inflammatory syndromes that lead to organ damage, and, in some cases, death. The virus can target the heart and blood vessels, but how it does it is a mystery to researchers. One study reported in the Journal of the American Medical Association that 20% of those in a Wuhan, China, study who were diagnosed with COVID-19 had damage to their heart muscle from the virus.

Here are some of the things researchers and doctors know about the virus they didn't know before:
It doesn't just affect the lungs.
It can cause blood clots.
It can attack children, causing an inflammatory response that can be deadly.
It can spread before people show symptoms of the disease.
It can cause a drop-in oxygen levels that should have left patients unconscious, yet they are still walking and talking.
The inflammation can cause skin rashes.
It attacks blood vessels.

Accordingly, it is desirable to have a platform that can automatically and intermittent or continuously monitoring of vital signs of patients. Monitoring of vital parameters most commonly includes at least body temperature, blood pressure and heart rate, and preferably also pulse oximetry and respiratory rate. Multimodal monitors that simultaneously measure and display the relevant vital parameters are commonly integrated into the bedside monitors in intensive care units and the anesthetic machines in operating rooms. These allow for continuous monitoring of a patient, with medical staff being continuously informed of the changes in general condition of a patient.

### Medical Records

The terms medical record, health record, and medical chart are used somewhat interchangeably to describe the systematic documentation of a single patient's medical history and care across time within one healthcare provider's jurisdiction. The medical record includes a variety of types of “notes” entered over time by health care professionals, recording observations and administration of drugs and therapies, orders for the administration of drugs and therapies, test results, x-rays, reports, etc. The maintenance of complete and accurate medical records is a requirement of health care providers and is generally enforced as a licensing or certification prerequisite.

Medical records have traditionally been compiled and maintained by health care providers, but advances in online data storage have led to the development of personal health records (PHR) that are maintained by patients themselves, often on third-party websites. This concept is supported by US national health administration entities and by AHIMA, the American Health Information Management Association.

In 2009, Congress authorized, and funded legislation known as the Health Information Technology for Economic and Clinical Health Act to stimulate the conversion of paper medical records into electronic charts. While many hospitals and doctor's offices have since done this successfully, electronic health vendors' proprietary systems haven't always been compatible with one another, and an untold number of patients undergo duplicate procedures—or fail to get them at all—because key pieces of their medical history are missing.

Because many consider the information in medical records to be sensitive private information covered by expectations of privacy, many ethical and legal issues are implicated in their maintenance, such as third-party access and appropriate storage and disposal. Although the storage equipment for medical records generally is the property of the health care provider, the actual record is considered in most jurisdictions to be the property of the patient, who can obtain copies upon request.

Large amounts of patient data are routinely manually collected in hospitals by using standalone medical devices, including vital signs. Such data is sometimes stored in spreadsheets, not forming part of patients' electronic health records, and is therefore difficult for caregivers to combine and analyze. One possible solution to overcome these limitations is the interconnection of medical devices via the Internet using a distributed platform, namely the Internet of Things. This approach allows data from different sources to be combined in order to better diagnose patient health status and identify possible anticipatory actions. It also helps make well-versed decisions and provide on-time treatment. Thus, IoT enables real-time alerting, tracking, and monitoring, which permits hands-on treatments, better accuracy, apt intervention by doctors and improve complete patient care delivery results.

Therefore, the ability to monitor vitals and track overtime and integrating this data across varying age, weight, gender, and overall health (pre-existing medical conditions) and lifestyle behaviors is critical to understanding, diagnosing and treating various diseases such as COVID-19.

### Electronic Health Records

An electronic health record (EHR) is the systematized collection of patient and population electronically stored health information in a digital format. These records can be shared across different health care settings. Records are shared through network-connected, enterprise-wide information systems or other information networks and exchanges. EHRs can include a range of data, including demographics, medical history, medication and allergies, immunization status, laboratory test results, radiology images, vital signs, personal statistics like age and weight, and billing information.

A decade ago, electronic health records (EHRs) were touted as key to increasing of quality care. Today, providers are using data from patient records to improve quality outcomes through their care management programs. Combining multiple types of clinical data from the system's health records has helped clinicians identify and stratify chronically ill patients. EHR can improve quality care by using the data and analytics to prevent hospitalizations among high-risk patients.

EHR systems are designed to store data accurately and to capture the state of a patient across time. It eliminates the need to track down a patient's previous paper medical records and assists in ensuring data is accurate and legible. It can reduce risk of data replication as there is only one modifiable file, which means the file is more likely up to date and decreases risk of lost paperwork. Due to the digital information being searchable and in a single file, EMRs (electronic medical records) are more effective when extracting medical data for the examination of possible trends and long-term changes in a patient. Population-based studies of medical records can also be facilitated by the widespread adoption of EHRs and EMRs.

### Remote Patient Monitoring

The patient monitoring platform can include a remote patient monitoring (RPM) technology, which is a technology that enables the monitoring of patients outside of conventional clinical settings, such as in the home or in a remote area. This can increase access to care and decrease healthcare delivery costs.

Incorporating RPM in chronic-disease management can significantly improve an individual's quality of life, by allowing patients to maintain independence, prevent complications, and to minimize personal costs. The patient monitoring platform with RPM facilitates these goals by delivering care through telecommunications. This form of patient monitoring can be particularly important when patients are managing complex self-care processes such as home hemodialysis. Key features of RPM, like remote monitoring and trend analysis of physiological parameters, enable early detection of deterioration; thereby reducing emergency department visits, hospitalizations, and the duration of hospital stays.

In some embodiments, the patient monitoring platform can employ RPM technologies and can have the following components in its RPM architecture.
Sensors on a device that is enabled by wireless communications to measure physiological parameters.
Local data storage at patients' site that interfaces between sensors and other centralized data repository and/or healthcare providers.
Centralized repository to store data sent from sensors, local data storage, diagnostic applications, and/or healthcare providers.
Diagnostic application software that develops treatment recommendations and intervention alerts based on the analysis of collected data.
Depending on the disease and the parameters that are monitored, different combinations of sensors, storage, and applications can be deployed.

### Biotelemetry

Biotelemetry (or medical telemetry) involves the application of telemetry in biology, medicine, and other health care to remotely monitor various vital signs of patients. The patient monitoring platform can include a biotelemetry system that includes:
Sensors for measuring and monitoring vital signs.
Battery-powered portable transceiver.
A display unit capable of concurrently presenting information from multiple patients.

### Telehealth & Telemedicine

Telehealth is the distribution of health-related services and information via electronic information and telecommunication technologies. It allows long-distance patient and clinician contact, care, advice, reminders, education, intervention, monitoring, and remote admissions. Telemedicine is sometimes used as a synonym or is used in a more limited sense to describe remote clinical services, such as diagnosis and monitoring. The Health Resources and Services Administration distinguishes telehealth from telemedicine in its scope, defining telemedicine only as describing remote clinical services, such as diagnosis and monitoring, while telehealth includes preventative, promotive, and curative care delivery. This includes the above-mentioned non-clinical applications,

When rural settings, lack of transport, a lack of mobility, decreased funding, or a lack of staff restrict access to care, telehealth can bridge the gap as well as provider distance-learning; meetings, supervision, and presentations between practitioners; online information and health data management and healthcare system integration. Telehealth could include two clinicians discussing a case over video conference; a robotic surgery occurring through remote access; physical therapy done via digital monitoring instruments, live feed and application combinations; tests being forwarded between facilities for interpretation by a higher specialist; home monitoring through continuous sending of patient health data; client to practitioner online conference; or even videophone interpretation during a consult.

### Population Health

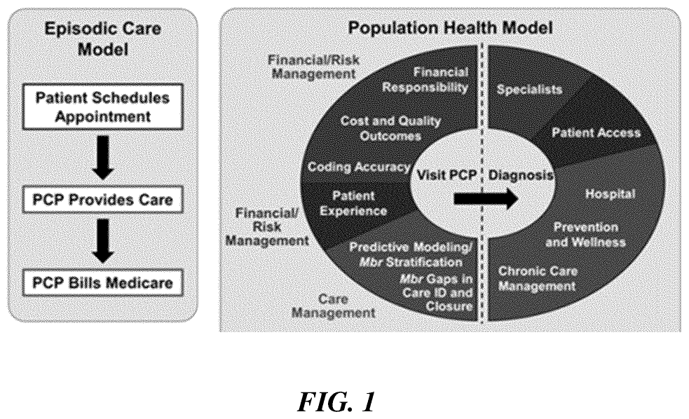

The concept of population health first came about in 2003 when David Kindig and Greg Stoddart defined it as “the health outcome of a group of individuals, including the distribution of such outcomes within the group.” FIG. 1 is a chart comparing the episodic care model and the population health model. As shown, the population health model includes three primary components: 1) health outcomes and their distribution with a population (morbidity, mortality and quality of life) 2) health determinants that influence distribution (medical care, socioeconomic status and genetics) 3) policies and interventions that influence these determinants (social, environmental and individual lifestyle behaviors). The key to innovation under the population health concept is evolving from episodic care to holistic care models.

### Internet of Things

The patient monitoring platform can include the internet of things (IOT) technology, which is a technology that interrelates computing devices, mechanical and digital machines provided with unique identifiers (UIDs) and the ability to transfer data over a network without requiring human-to-human or human-to-computer interaction.

The patient monitoring platform leverages multiple technologies such as real-time analytics, machine learning, commodity sensors, and embedded systems. Traditional fields of embedded systems, wireless sensor networks, control systems, automation (including home and building automation), and others all contribute to enabling IoTs.

### Internet of Medical Things

The patient monitoring platform also leverages IoMT technology, which is a technology that enables connected infrastructure of medical devices, software applications, and health systems and services.

And while a growing pool and general adoption of IoT technologies are benefiting many industries, it is a wave of sensor-based tools—including wearables and stand-alone devices for remote patient monitoring—and the marriage of internet-connected medical devices with patient information that ultimately set the IoMT ecosystem apart.

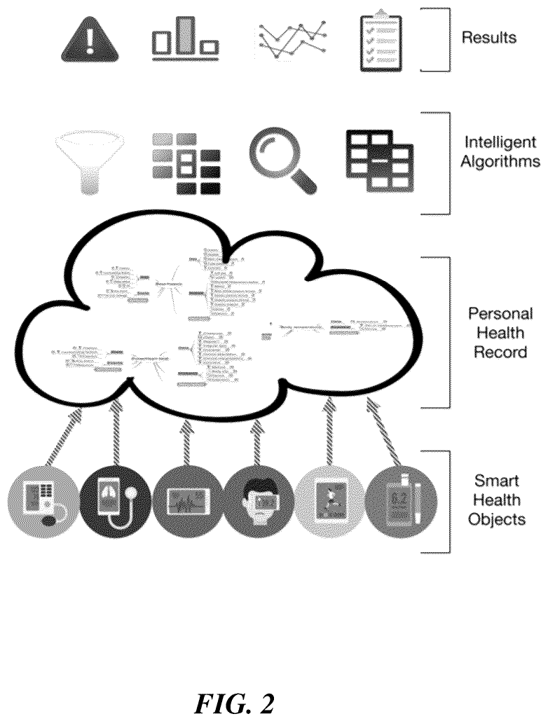

Large amounts of patient data are routinely manually collected in hospitals by using standalone medical devices, including vital signs. Such data is sometimes stored in spreadsheets, not forming part of patients' electronic health records, and is therefore difficult for caregivers to combine and analyze. To overcome these limitations, the patient monitoring platform leverages the interconnection of medical devices via the Internet using a distributed platform, namely the Internet of Things. This approach allows data from different sources to be combined in order to better diagnose patient health status and identify possible anticipatory actions. FIG. 2 graphically illustrates the implementation of IoMT by the patient monitoring platform to collect and access the PHR and diagnosis using various ML algorithms.

It also helps make well-versed decisions and provide on-time treatment. Thus, the patient monitoring platform with IoT enables real-time alerting, tracking, and monitoring, which permits hands-on treatments, better accuracy, apt intervention by doctors and improve complete patient care delivery results.

### Regulatory Compliance

GDPR—The General Data Protection Regulation (EU) 2016/679 (GDPR) is a regulation in EU law on data protection and privacy in the European Union (EU) and the European Economic Area (EEA). It also addresses the transfer of personal data outside the EU and EEA areas. The GDPR aims primarily to give control to individuals over their personal data and to simplify the regulatory environment for international business by unifying the regulation within the EU. Superseding the Data Protection Directive 95/46/EC, the regulation contains provisions and requirements related to the processing of personal data of individuals (formally called data subjects in the GDPR) who are located in the EEA, and applies to any enterprise—regardless of its location and the data subjects' citizenship or residence—that is processing the personal information of data subjects inside the EEA.

Controllers and processors of personal data must put in place appropriate technical and organizational measures to implement the data protection principles. Business processes that handle personal data must be designed and built with consideration of the principles and provide safeguards to protect data (for example, using pseudonymization or full anonymization where appropriate). Data controllers must design information systems with privacy in mind, for instance use the highest-possible privacy settings by default, so that the datasets are not publicly available by default and cannot be used to identify a subject. No personal data can be processed unless this processing is done under one of six lawful bases specified by the regulation (consent, contract, public task, vital interest, legitimate interest or legal requirement). When the processing is based on consent the data subject has the right to revoke it at any time.

PIPEDA—The Personal Information Protection and Electronic Documents Act (PIPEDA) is a Canadian law relating to data privacy. It governs how private sector organizations collect, use and disclose personal information in the course of commercial business. In addition, the Act contains various provisions to facilitate the use of electronic documents. PIPEDA became law on 13 Apr. 2000 to promote consumer trust in electronic commerce. The act was also intended to reassure the European Union that the Canadian privacy law was adequate to protect the personal information of European citizens. In accordance with section 29 of PIPEDA, Part I of the Act (“Protection of Personal Information in the Private Sector”) must be reviewed by Parliament every five years. The first Parliamentary review occurred in 2007.

PIPEDA incorporates and makes mandatory provisions of the Canadian Standards Association's Model Code for the Protection of Personal Information, developed in 1995. However, there are a number of exceptions to the Code where information can be collected, used and disclosed without the consent of the individual. Examples include reasons of national security, international affairs, and emergencies. Under the Act, personal information can also be disclosed without knowledge or consent to investigations related to law enforcement, whether federal, provincial or foreign. There are also exceptions to the general rule that an individual shall be given access to his or her personal information. Exceptions can include information that would likely reveal personal information about a third party, information that cannot be disclosed for certain legal, security, or commercial proprietary reasons, and information that is subject to solicitor-client privilege.

HIPAA—The Health Insurance Portability and Accountability Act of 1996 (HIPAA or the Kennedy-Kassebaum Act was enacted by the 104th United States Congress and signed by President Bill Clinton in 1996. It was created primarily to modernize the flow of healthcare information, stipulate how Personally Identifiable Information maintained by the healthcare and healthcare insurance industries should be protected from fraud and theft, and address limitations on healthcare insurance coverage.

The act consists of five titles. Title I of HIPAA protects health insurance coverage for workers and their families when they change or lose their jobs. Title II of HIPAA, known as the Administrative Simplification (AS) provisions, requires the establishment of national standards for electronic health care transactions and national identifiers for providers, health insurance plans, and employers.[5] Title III sets guidelines for pre-tax medical spending accounts, Title IV sets guidelines for group health plans, and Title V governs company-owned life insurance policies.

PHIPA—The Personal Health Information Protection Act, (the Act) also known as PHIPA (‘pee-hip-ah’), is Ontario legislation established in November 2004. PHIPA is one of two components of the Health Information Protection Act. The Health Information Protection Act, also established in 2004, comprises two schedules: PHIPA (Schedule A) and the Quality of Care Information Protection Act (Schedule B.

PHIPA provides a set of rules for the collection, use and disclosure of personal health information, and includes the following provisions:
Consent is required for the collection, use and disclosure of personal health information, with few exceptions.
Health information custodians are required to treat all personal health information as confidential and maintain its security.
Individuals have a right to access their personal health information, as well as the right to correct errors.
Individuals have the right to instruct health information custodians not to share their personal health information with others.
Rules are provided for the use of personal health information for fundraising or marketing purposes.
Guidelines are set for the use and disclosure of personal health information for research purposes.
Accountability is ensured by granting an individual the right to complain if they have identified an error in their personal health information.
Remedies are established for breaches of the legislation.

HITECH—The Health Information Technology for Economic and Clinical Health Act, abbreviated HITECH Act, was enacted under Title XIII of the American Recovery and Reinvestment Act of 2009 (Pub.L. 111-5). Under the HITECH Act, the United States Department of Health and Human Services (U.S.HHS) resolved to spend $25.9 billion to promote and expand the adoption of health information technology.

Health information exchange (HIE) has emerged as a core capability for hospitals and physicians to achieve “meaningful use” and receive stimulus funding. Starting in 2015, hospitals and doctors will be subject to financial penalties under Medicare if they are not using electronic health records.

### Meaningful Use

The main components of meaningful use are:
The use of a certified EHR in a meaningful manner, such as e-prescribing.
The use of certified EHR technology for electronic exchange of health information to improve quality of health care.
The use of certified EHR technology to submit clinical quality and other measures.

In other words, providers need to show they're using certified EHR technology in ways that can be measured significantly in quality and in quantity.

The meaningful use of EHRs intended by the US government incentives is categorized as follows:
Improve care coordination.
Reduce healthcare disparities.
Engage patients and their families.
Improve population and public health.
Ensure adequate privacy and security.

FDA CLASS II, 510(k)—On Can 28, 1976, the FD&C Act was amended to include regulation for medical devices. The amendment required that all medical devices be classified into one of three classes:
Class I: Devices that do not require premarket approval or clearance but must follow general controls. Dental floss is a class I device.
Class II: Devices that are cleared using the 510(k) process. Diagnostic tests, cardiac catheters, hearing aids, and dental amalgams are examples of class II devices.
Class III: Devices that are approved by the Premarket Approval (PMA) process, analogous to a New Drug Application. These tend to be devices that are permanently implanted into a human body or can be necessary to sustain life. An artificial heart meets both criteria. The most commonly recognized class III device is an Automated External Defibrillator. Devices that do not meet either criterion are generally cleared as class II devices.

For devices that were marketed prior to the amendment (Preamendment devices) and were classified as Class III, the amendment obligated the FDA to review the device to either reclassify it as a Class II device subject to premarket notification, or to require the device manufacturer to undergo the premarket authorization process and prove the safety and efficacy of the device in order to continue marketing it. Notable examples of such preamendment devices are those used for electroconvulsive therapy, which the FDA started reviewing in 2011.

Premarket Notification (510(k), PMN)

Section 510(k)[24] of the Federal Food, Drug, and Cosmetic Act requires those device manufacturers who must register to notify FDA, at least 90 days in advance, of their intent to market a medical device.

This is known as Premarket Notification, PMN, or 510(k). It allows FDA to determine whether the device is equivalent to a device already placed into one of the three classification categories. Thus, “new” devices (not in commercial distribution prior to Can 28, 1976) that have not been classified can be properly identified.

Any device that reaches market via a 510(k) notification must be “substantially equivalent” to a device on the market prior to Can 28, 1976 (a “predicate device”). If a device being submitted is significantly different, relative to a pre-1976 device, in terms of design, material, chemical composition, energy source, manufacturing process, or intended use, the device nominally must go through a premarket approval, or PMA. This does not always happen.

A device that reaches market via the 510(k) process is not considered to be “approved” by the FDA. Nevertheless, it can be marketed and sold in the United States. They are generally referred to as “cleared” or “510(k) cleared” devices.

A 2011 study by Dr. Diana Zuckerman and Paul Brown of the National Research Center for Women and Families, and Dr. Steven Nissen of the Cleveland Clinic, published in the Archives of Internal Medicine, showed that most medical devices recalled in the last five years for “serious health problems or death” had been previously cleared by the FDA using the less stringent, and cheaper, 510(k) process. In a few cases the devices had been deemed so low-risk that they did not need FDA regulation. Of the 113 devices recalled, 35 were for cardiovascular issues. This can lead to a reevaluation of FDA procedures and better oversight.

The patient monitoring platform is designed to provide one or more of following features (non-exhaustive list):
Accessibility—low-cost
Frictionless, ease of use—User Interface Design and User Experience Design (UI/UX)
Private, Secure and Compliant
Privacy, Encryption and Compliance (GDPR, PIPEDA, HIPAA, PHIPA, HITECH, FDA Class II 510K)
User identification and authentication (biometrics, facial recognition, time stamping)
Device authentication and self-diagnostics
Data—sharing and integration; tracking and analytics; machine learning for diagnosis and predictive health outcomes.
Artificial Intelligence
   Interactive AI—Conversational AI (chat bots and smart personal assistants)
   Functional AI—Internet of Medical Things
   Visual AI—Computer vision (facial recognition, geo-location and timestamping)
   Analytic AI—Machine learning
Process for COVID-19:
   social Distancing and hygiene education,
   routine, daily vitals checks
   contact tracing
   antibody and PCR testing

### Privacy, Encryption and Compliance (GDPR & HIPAA)

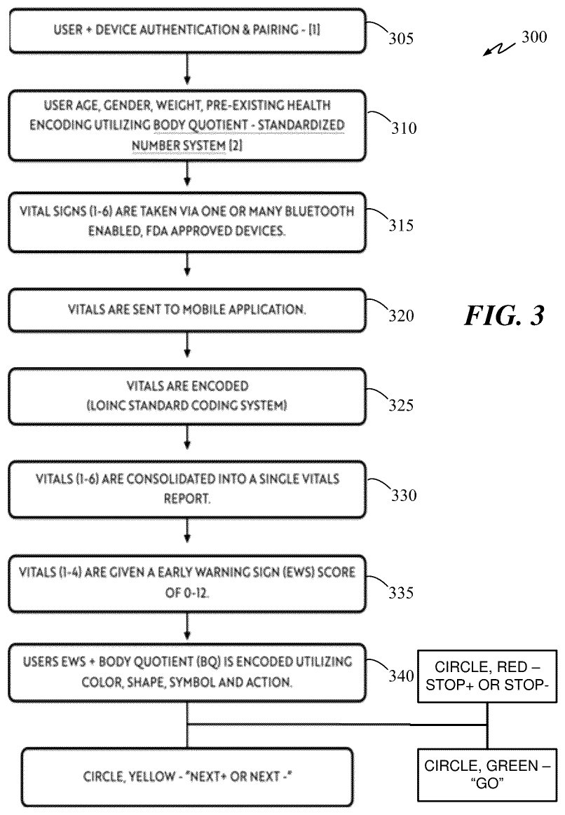

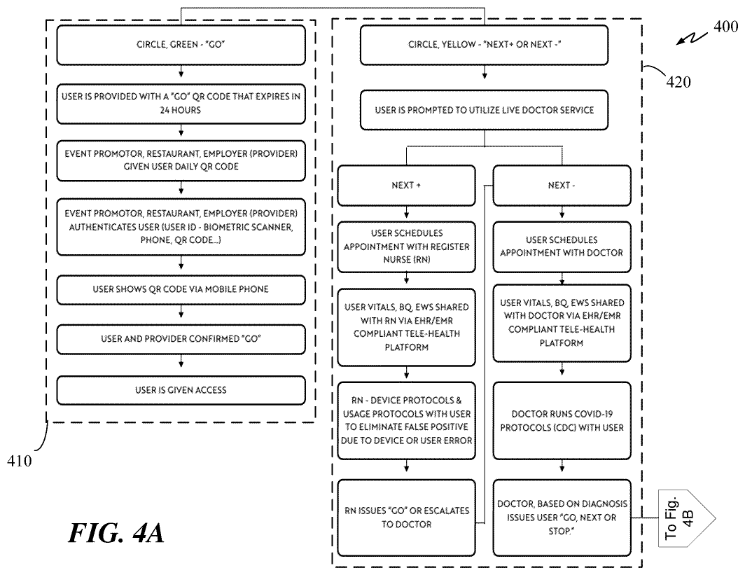

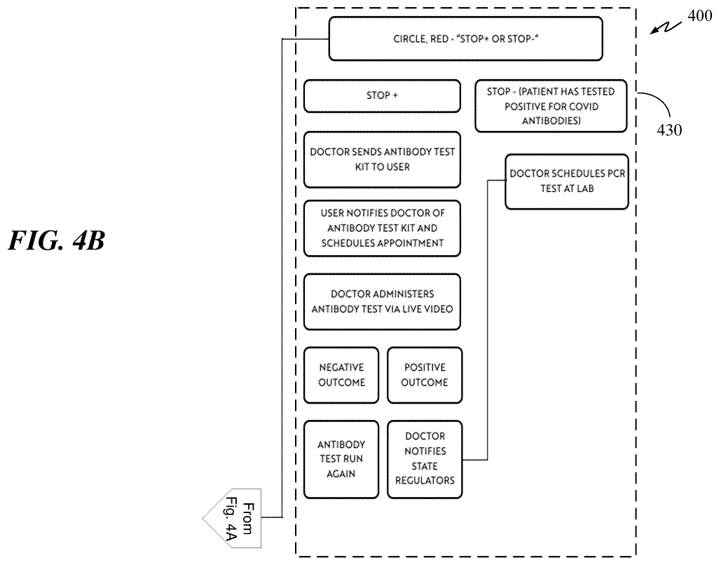

In some embodiments, the patient monitoring platform is configured to be HIPAA & GDPR compliant with respect to data handling such as sharing of vitals of individuals for public health purposes. FIGS. 3, 4A and 4B illustrate processes 300 and 400 for sharing of vitals of individuals under the compliance of HIPAA & GDPR in accordance with some embodiments of the disclosure. The patient monitoring platform is configured to implement process 300 and/or 400 in order to conform with the HIPAA & GDPR requirements.

Process 300 starts at 305 where one or more of the user's devices is authenticated. At 310, the user's age, gender, weight, and pre-existing condition are encoded using the body quotient standard number system. At 315, vital signs are taken via one or more Bluetooth enabled devices. At 320, vitals signs are sent to mobile application. At 325, vital signs are encoded using the LOINC standard. At 330, vital signs are consolidated into a single report. At 335, vital signs are given an EWS score. At 340, the user's EWS and/or body quotient is encoded using color, shape symbol, and/or action. A yellow circle can mean next. A circle red can mean stop. A circle green can mean go. It should be noted although GREEN, YELLOW and RED indicia are used throughout the present disclosure, other suitable indicia can also be possible. FIGS. 4A and 4B illustrate process 400 for the three different conditions: Circle, Green—GO (see process group 410); Circle, Yellow—Next (see process group 420); and Circle, Red Stop (see process group 430).

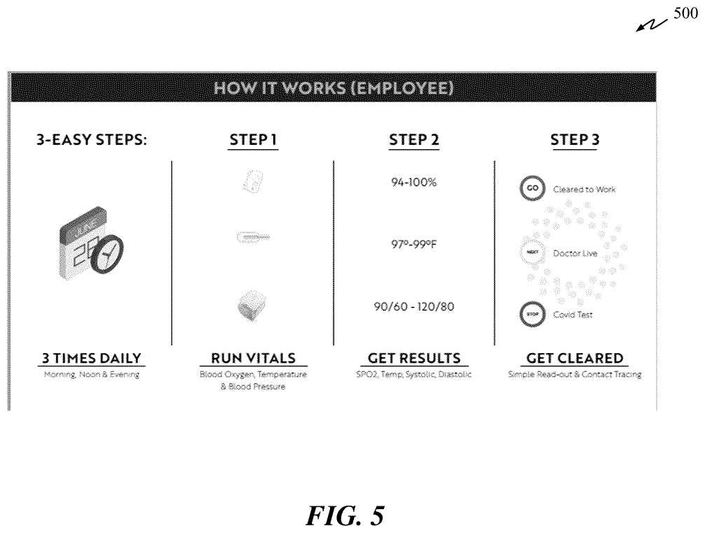

FIG. 5 illustrates a process 500 for a person (e.g., employee) to follow to receive a clearance for an activity (e.g., return to work) or for further treatments in accordance with some embodiments of the disclosure. Process 500 can include three steps that can be performed several times each day (e.g., three times daily). In step 1, the employee can run the vitals, such as Blood Oxygen, Temperature & Blood Pressure, using the devices of the present disclosure. In step 2, the devices provide the results for the vitals, for example, SPO2, temperature, Systolic, Diastolic, etc. In step 3, the devices can provide simple read-out & contact tracing information. For example, the read out can include color coded information: Green denotes employee is healthy and cleared for the designated premise (e.g., go to work), Yellow denotes employee needs to be referred to a live doctor, and Red denotes the employee needs to obtain further test, e.g., a COVID-19 test. In some embodiments, patient monitoring platform 600 can include process 500 to perform clearance for an activity or obtain approval for further and/or different treatments.

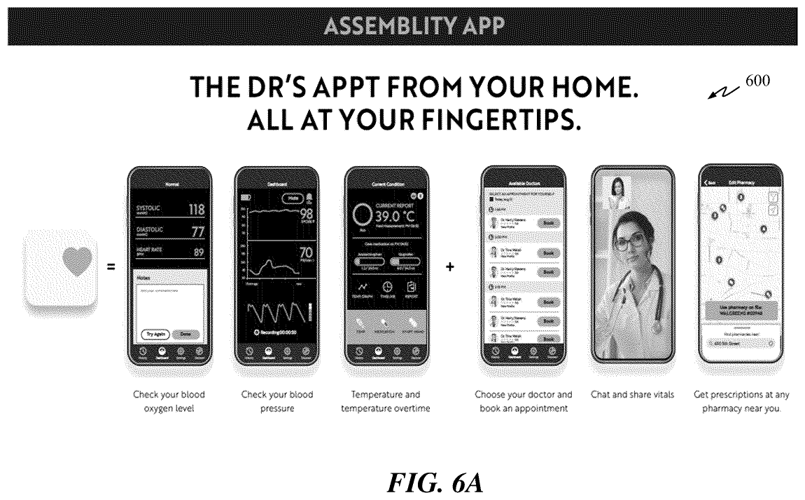

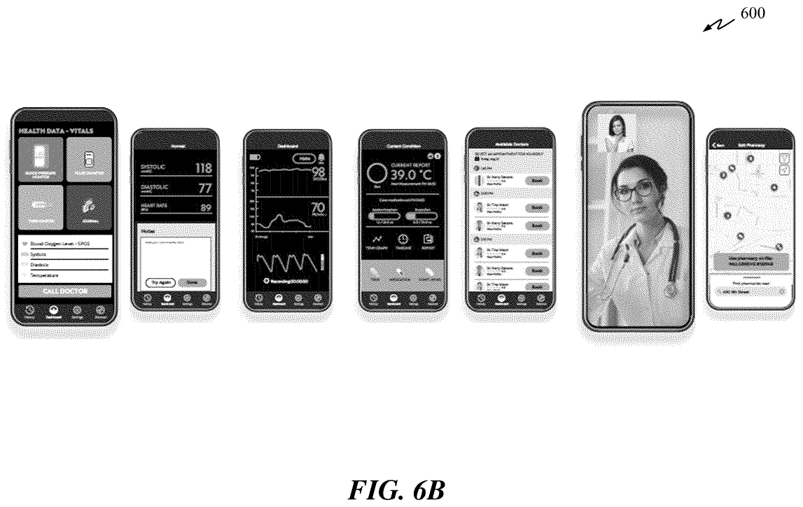

FIGS. 6A and 6B illustrate various application of patient monitoring platform 600 in accordance with some embodiments of the disclosure. Patient monitoring platform 600 can include a health application 605 that can reside on a mobile device. Health application 605 can receive data from the devices and connect the user to a live doctor via a network (not shown) such as a local area network connected to the internet. Health application 605 can be connected to a pharmacy, an insurance company, etc., as needed for data exchanges. Health App 600 can include one or more features configured to check your blood oxygen level, blood pressure, temperature, appointments, chat room, and nearby pharmacy (see FIGS. 6A and 6B).

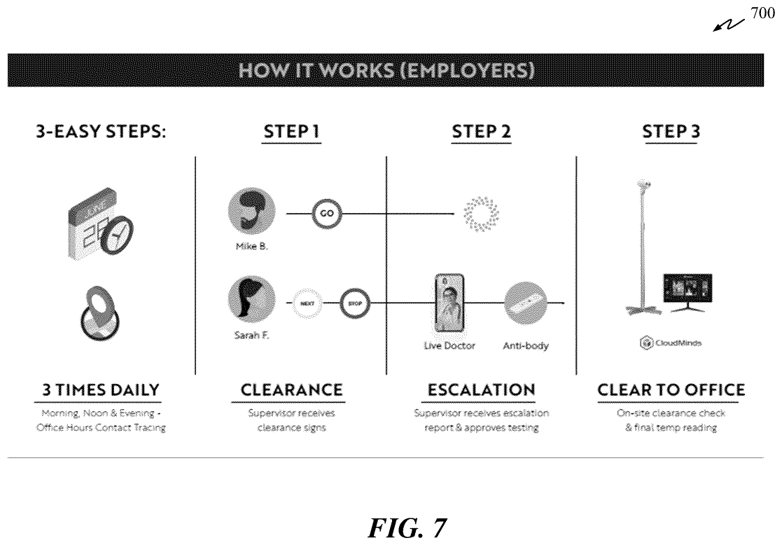

FIG. 7 illustrates a process 700 that can provide clearance for another user for an activity (e.g., return to work) or further treatments in accordance with some embodiments. Process 700 can include three steps that can be performed several times each day (e.g., three times daily). In step 1, the employer can receive, from the system and devices, simple read-out of Green, Yellow or Red as described above for an employee. Or the employer can receive progress, for example, the employee progresses from Yellow to Red or Green. In step 2, based on the read-out report, the system and devices can clear the employee for the activity, refer the employee to a live doctor or to further test. In step 3, at the office or an event, the employer can use the system and devices to clear and authenticate the employee.

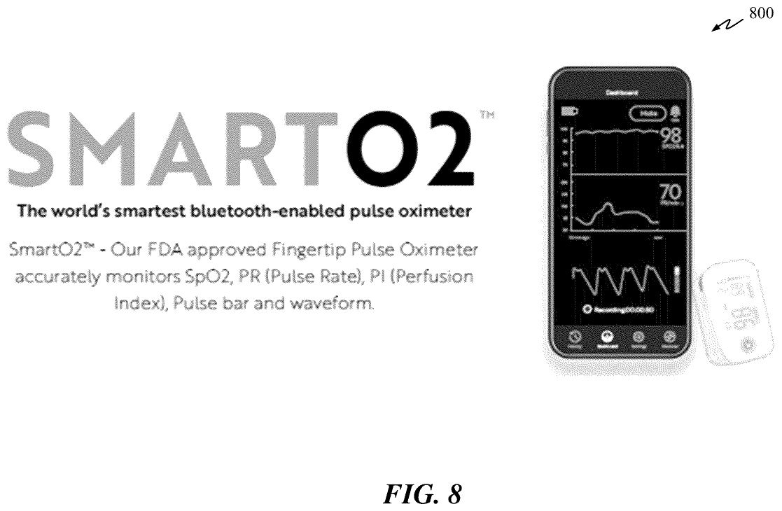

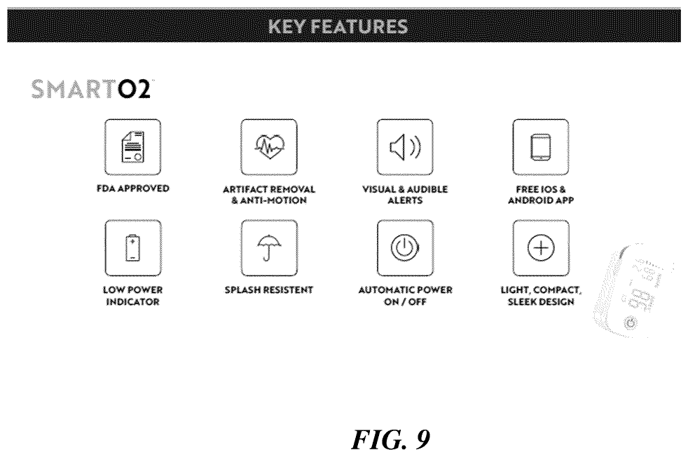

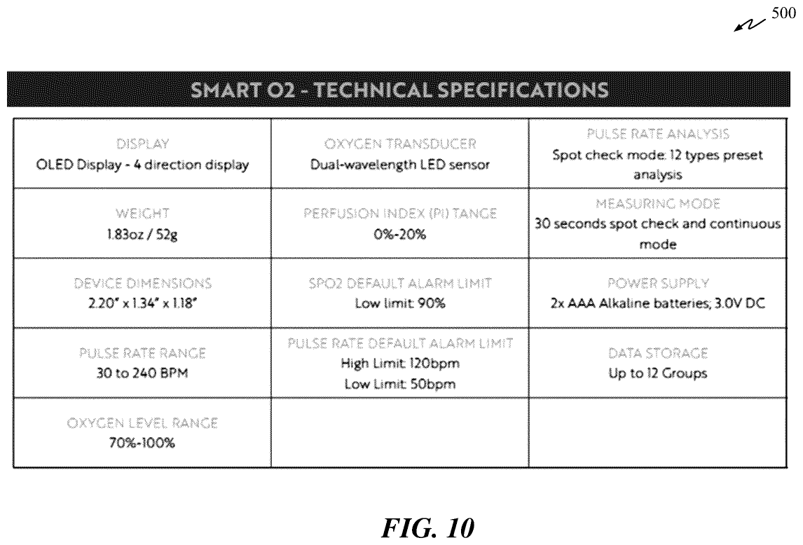

FIG. 8 illustrates a smart Bluetooth-enabled pulse oximeter 800, which can be one of the devices on the patient monitoring platform. FIG. 9 shows exemplary features of the pulse oximeter 800. Pulse oximeter 800 can include accurate fingertip monitoring of at least SpO2 (peripheral capillary oxygen saturation), PR (Pulse Rate), PI (Perfusion Index), and pulse bar. In some embodiments, Pulse oximeter 800 can automatically power on and start measurement when placed on a user's finger. Pulse oximeter 800 can store at least 12 groups of SpO2 data in the on-board memory, with full-history available on a mobile app. Pulse oximeter 800 can also automatically provide visual and audible alerts when low oxygen levels and/or abnormal heartbeat is detected. FIG. 10 illustrates some of the technical specifications of pulse oximeter 800, according to some embodiments of the present disclosure.

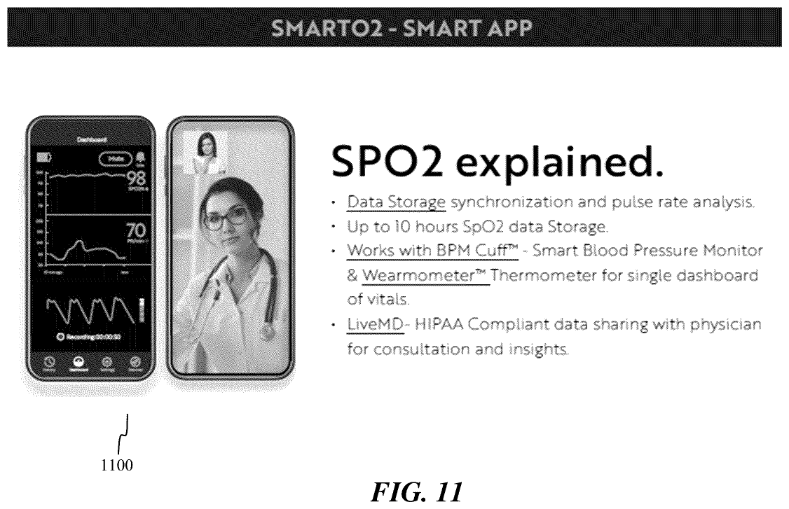

FIG. 11 illustrates a screen shot of a SPO2 app 1100 that can be part of the patient monitoring platform in accordance with some embodiments of the disclosure. SPO2 app 1100 can include data storage synchronization and pulse rate analysis, at least 10 hours SpO2 data storage. It can work with BPM Cuff™—Smart Blood Pressure Monitor & Wearmometer™ Thermometer for single dashboard of vitals. It can also include LiveMD—HIPAA Compliant data sharing with physician for consultation and insights.

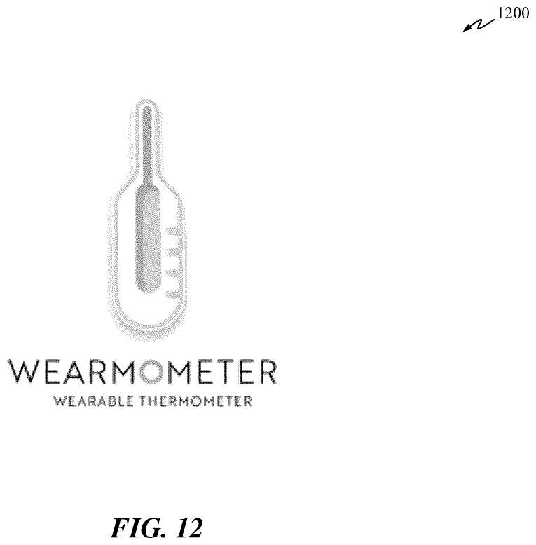

FIG. 12 illustrates a “wearmometer” 1200 (wearable thermometer) that can be part of the patient monitoring platform in accordance with some embodiments of the present disclosure. Wearmometer 1200 can accurately monitor temperature with a simple tap on a mobile phone. Wearmometer 1200 can easily track the wearer's temperature over time. It can use no electromagnetic waves, is reusable and washable.

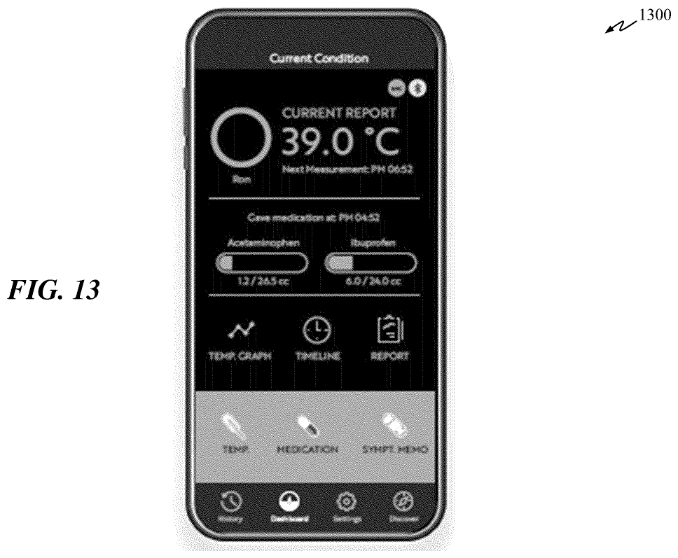

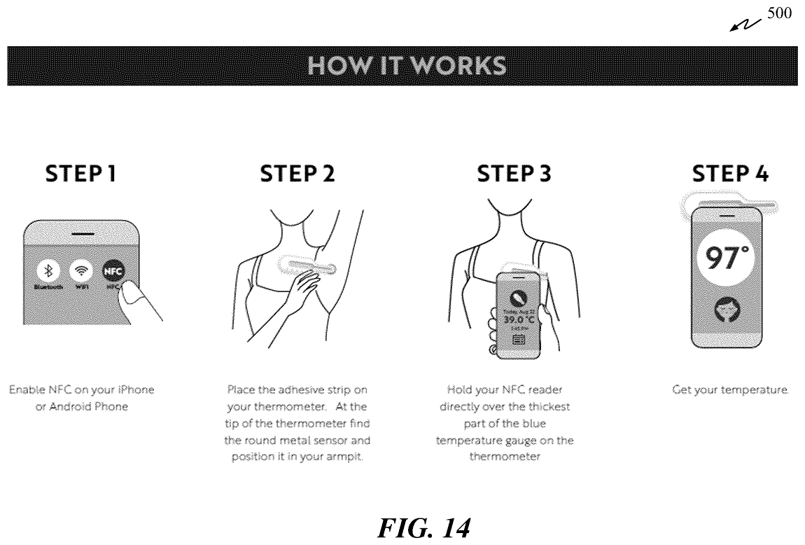

FIG. 13 illustrates a wearmometer app 1300 that can be paired with wearmometer 1200. App 1300 can be part of the patient monitoring platform. App 1300 can provide a complete picture of the user's health by encouraging users to input all symptoms and an algorithm can take care of the rest. The system and device can combine core vitals with any symptoms and medication to have a few views of the user's illness.

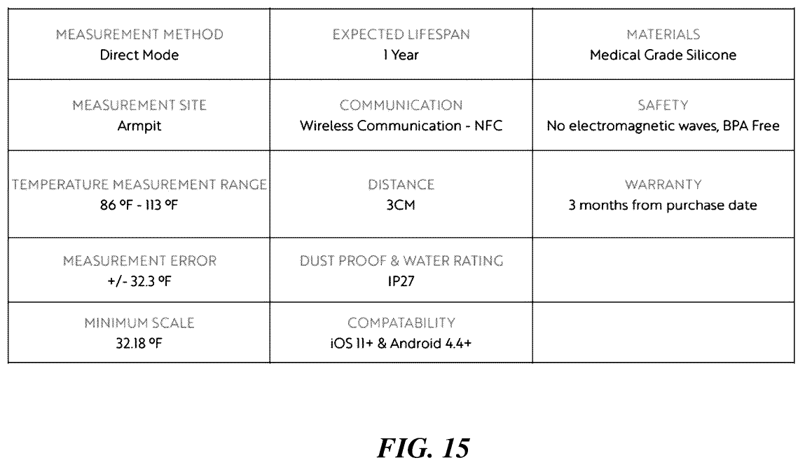

FIG. 14 illustrates an exemplary process for using wearable thermometer 1200 with the app 1300 in accordance with some embodiments of the present disclosure. For example, the NFC on the user's mobile phone (e.g., iPhone or Android Phone) can first be activated or enabled. The user can then place a provided adhesive strip on the thermometer. At the tip of the thermometer is a round metal sensor which when positioned in a reading location, e.g., user's armpit, will read the user's temperature. When the NFC reader (mobile phone) is positioned directly over a thickest part of the blue temperature gauge on the thermometer, app 1300 receives and displays the user's temperature. FIG. 15 is a table providing some exemplary specifications of wearable thermometer 1200, according to some embodiments of the present disclosure.

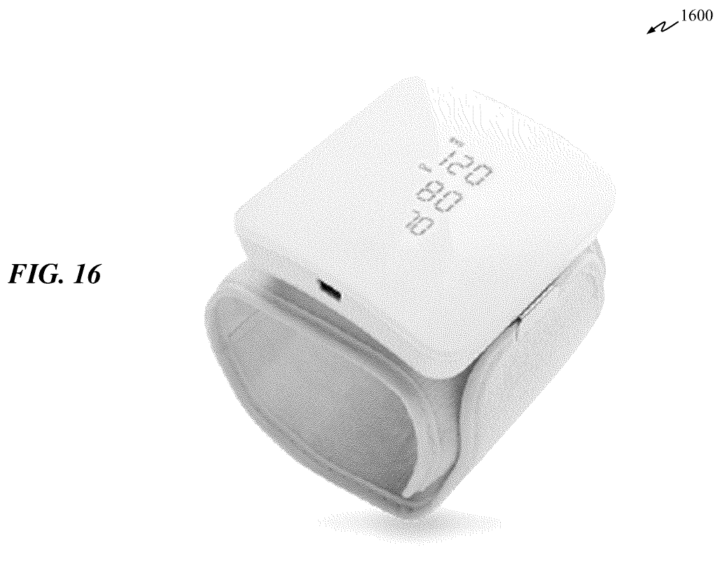

FIG. 16 illustrates a wearable blood pressure monitor 1600 that can be part of the patient monitoring platform in accordance with some embodiments. Blood pressure monitor 1600 can be a smart cuff configured to accurately measure and monitor a user's blood pressure over time. With a press of a button, results appear right on a screen. It can also provide automatic Wi-Fi sync. Blood pressure monitor 1600 is lightweight, breathable and collapsible to take on the go.

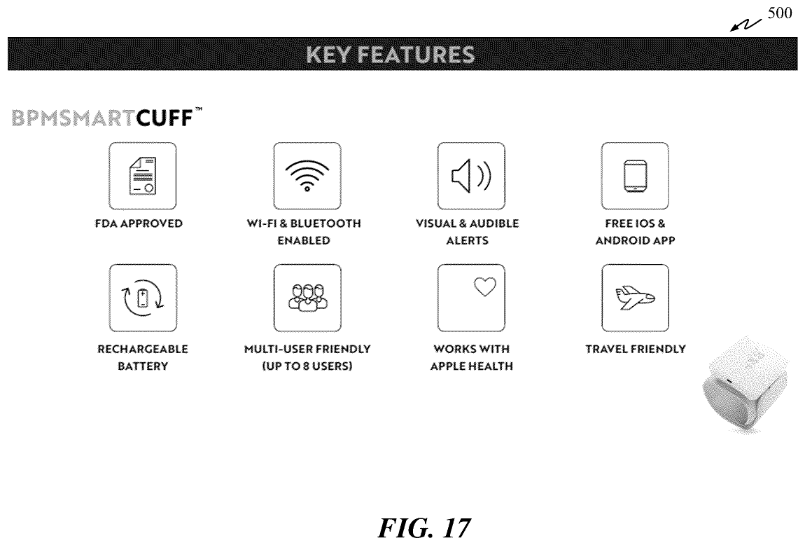

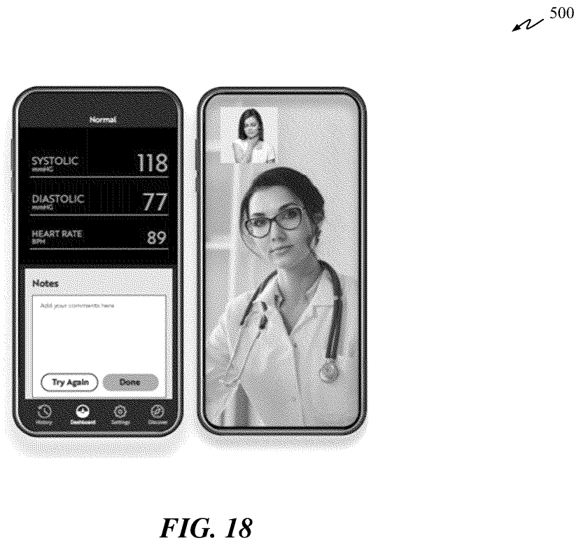

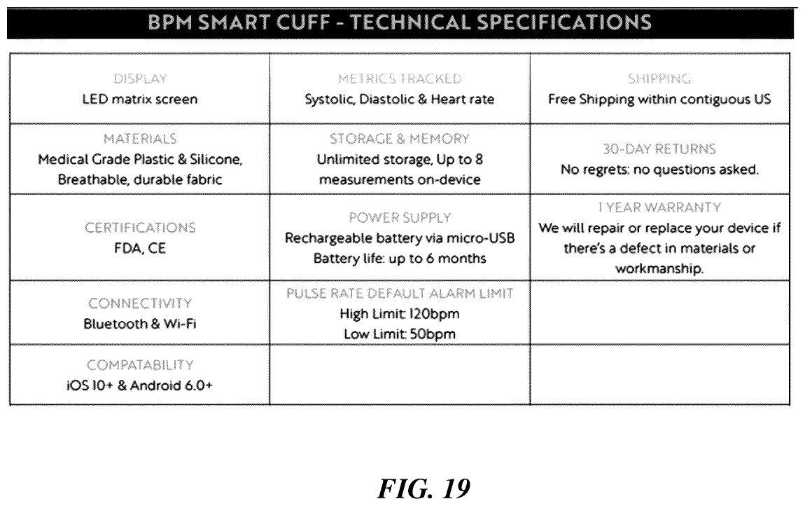

FIGS. 17 and 18 illustrate features and screenshot of the wearable blood pressure monitor 1600, according to some embodiments of the present disclosure. For example, it can sync, e.g., via Wi-Fi and/or Bluetooth with a free Revival Health app, available for iOS or Android, or other OS. With Wi-Fi synchronization, there can be no need to have the smartphone next to the user during or after the measurement, as data is automatically synchronized via a Wi-Fi network (e.g., home network). A corresponding app can provide unlimited storage. FIG. 19 is a table showing some exemplary specifications and features of the wearable blood pressure monitor 1700, according to some embodiments of the present disclosure.

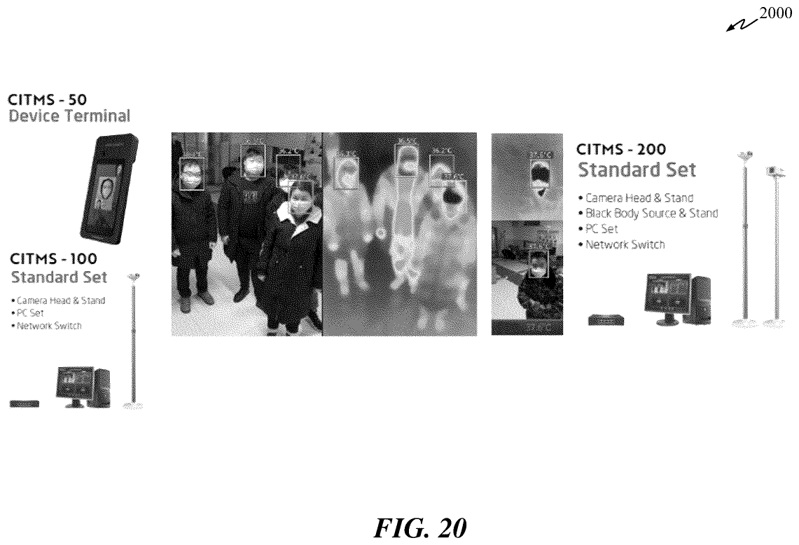

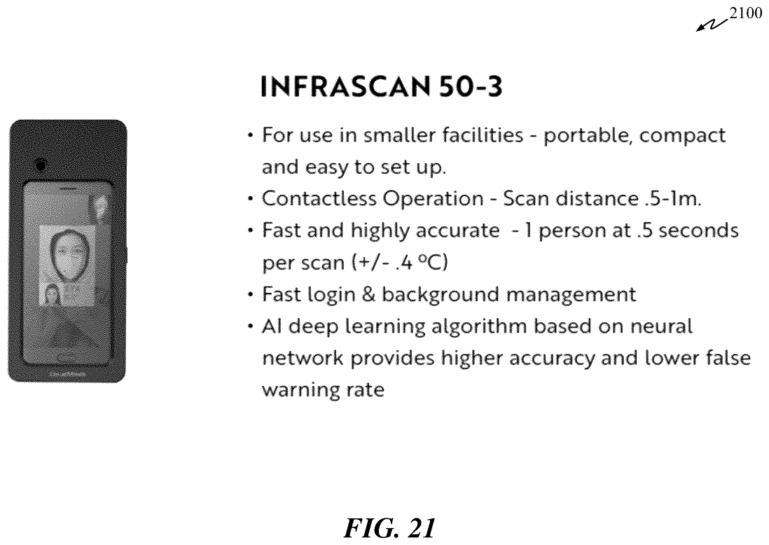

The health monitoring platform can include infrared thermal scanners 2000 as shown in FIGS. 20 and 21, which can be referred to as InfraScan. In some embodiments, infrared thermal scanners 2000 can include the authentication features and capabilities as described in PCT Patent Application No. PCT/US20/25840, the disclosure of which is hereby incorporated by reference in its entirety. Infrared thermal scanners 200 can authenticate the users and the same persons whose vitals were taken and stored either in the devices and/or in the cloud. In some embodiments, biometric data and QR code can be included or used.

FIG. 21 illustrates an exemplary handheld infrared thermal scanner 2100 in accordance with some embodiments of the present disclosure. Handheld infrared thermal scanner 2100 can be used in smaller facilities, as it is portable compact and easy to set up. Handheld infrared thermal scanner 2100 can include contactless operation (e.g., with scan distance about 0.5 to 1.0 meter). Handheld infrared thermal scanner 2100 is fast and highly accurate, e.g., it can scan—1 person at about 0.5 seconds per scan (+/−0.4° C.). In some embodiments, handheld infrared thermal scanner 2100 can include a fast login and background management features. It can further include AI deep learning algorithm based on neural network provides higher accuracy and lower false warning rate. It should be noted that infrared thermal scanners 2000 can include one or more features of infrared thermal scanners 2100.

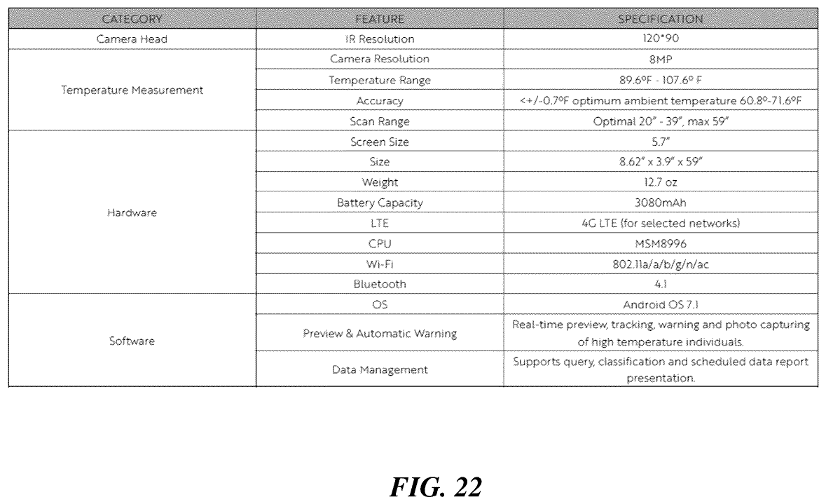

FIG. 22 is a table showing some exemplary specifications of handheld infrared thermal scanner 2000 and/or 2100, according to some embodiments of the present disclosure.

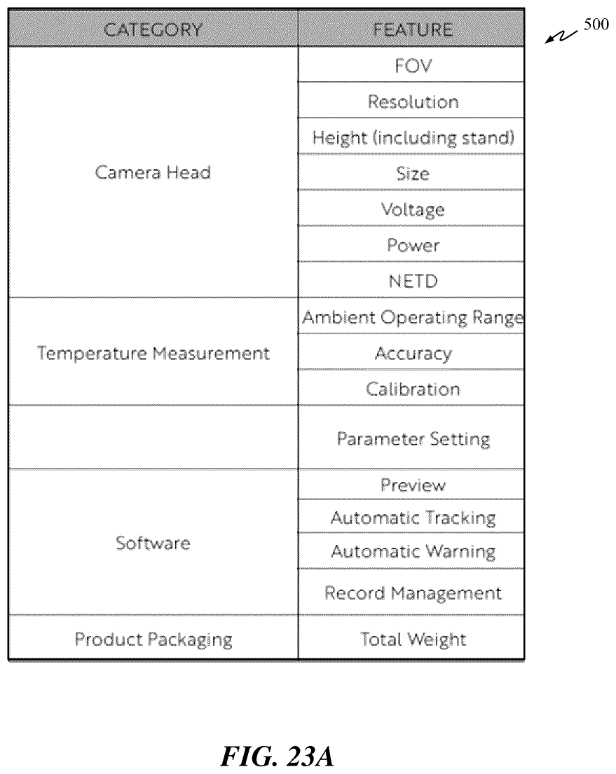

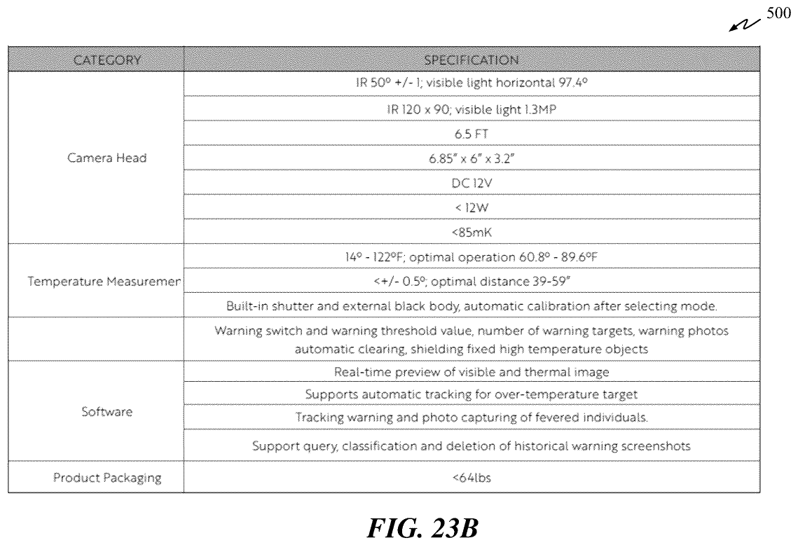

Infrared thermal scanner 2000 and/or 2100 can have technical specifications as shown in FIGS. 23A and 23B. Infrared thermal scanner 2000 and/or 2100 can be used with crowds and include customizable privacy controls stored locally or via ultra-high security cloud. In some embodiments, infrared thermal scanner 2000 and/or 2100 can provide accurate single-point and multi-point high temperature with detection of up to 3 people simultaneously. Infrared thermal scanner 2000 and/or 2100 can also include AI deep learning algorithm based on neural network for accuracy and lower false warning rate. In some embodiments, infrared thermal scanner 2000 and/or 2100 can utilize high-end 120×90 infrared uncooled Vanadium Oxide (Vox) detector.

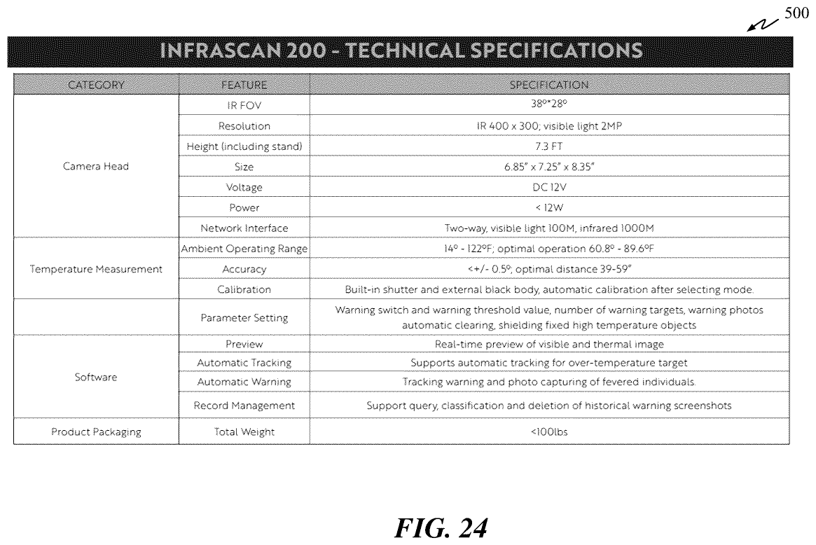

Infrared thermal scanner 2000 and/or 2100 can have technical specifications as shown in FIG. 24. Based on the specification of FIG. 24, infrared thermal scanner 2000 and/or 2100 can provide accurate single-point and multi-point high temperature with detection of up to 8 people simultaneously. Higher detection can also be possible. infrared thermal scanner 2000 and/or 2100 can also include AI deep learning algorithm based on neural network for accuracy and lower false warning rate. In some embodiments, infrared thermal scanner 2000 and/or 2100 can utilize at least high-end 400×300 infrared uncooled Vox detector.

It should be recognized that while the present disclosure refers to using a “mobile” device and “mobile” application to perform certain functions disclosed herein, any of the functions performed by such mobile devices or applications can alternatively, or additionally, be performed by other types of computing devices (e.g., desktop computers, laptop computers, etc.) and/or applications installed thereon. Thus, any function or use of the mobile devices and/or mobile applications disclosed herein can also be executed by other types of computer devices and/or applications installed thereon.

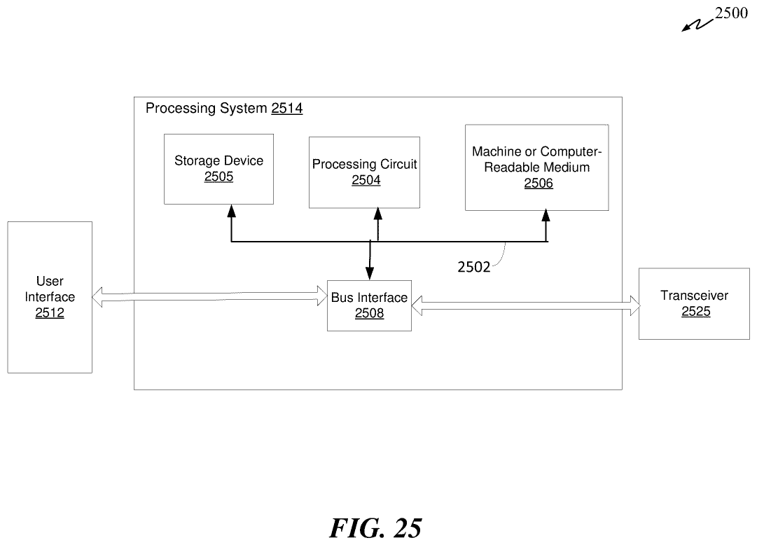

FIG. 25 illustrates an exemplary overall platform 2500 in which various embodiments and process steps disclosed herein can be implemented. In accordance with various aspects of the disclosure, an element, or any portion of an element, or any combination of elements can be implemented with a processing system 2514 that includes one or more processing circuits 2504. Processing circuits 2504 can include micro-processing circuits, microcontrollers, digital signal processing circuits (DSPs), field programmable gate arrays (FPGAs), programmable logic devices (PLDs), state machines, gated logic, discrete hardware circuits, and other suitable hardware configured to perform the various functionalities described throughout this disclosure, including the process steps, GUIs, and features of technical specification as illustrated at least in FIGS. 2 through 24. That is, the processing circuit 2504 can be used to implement any one or more of the various embodiments, systems, algorithms, and processes described above. In some embodiments, the processing system 2514 can be implemented in a server. The server can be local or remote, for example in a cloud architecture.

In the example of FIG. 25, the processing system 2514 can be implemented with a bus architecture, represented generally by the bus 2502. The bus 2502 can include any number of interconnecting buses and bridges depending on the specific application of the processing system 2514 and the overall design constraints. The bus 2502 can link various circuits including one or more processing circuits (represented generally by the processing circuit 2504), the storage device 2505, and a machine-readable, processor-readable, processing circuit-readable or computer-readable media (represented generally by a non-transitory machine-readable medium 2506). The bus 2502 can also link various other circuits such as timing sources, peripherals, voltage regulators, and power management circuits, which are well known in the art, and therefore, will not be described any further. The bus interface 2508 can provide an interface between bus 2502 and a transceiver 2510. The transceiver 2510 can provide a means for communicating with various other apparatus over a transmission medium. Depending upon the nature of the apparatus, a user interface 2512 (e.g., keypad, display, speaker, microphone, touchscreen, motion sensor) can also be provided.

The processing circuit 2504 can be responsible for managing the bus 2502 and for general processing, including the execution of software stored on the machine-readable medium 2506. The software, when executed by processing circuit 2504, causes processing system 2514 to perform the various functions described herein for any apparatus. Machine-readable medium 2506 can also be used for storing data that is manipulated by processing circuit 2504 when executing software.

One or more processing circuits 2504 in the processing system can execute software or software components. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. A processing circuit can perform the tasks. A code segment can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory or storage contents. Information, arguments, parameters, data, etc. can be passed, forwarded, or transmitted via any suitable means including memory sharing, message passing, token passing, network transmission, etc.

It should be understood that exemplary embodiments described above are not intended to be limiting and that the inventive systems, methods, apparatuses, computer program products, and techniques described herein can be used in many other scenarios as well. It should also be further understood that the configurations and structures of the system components can vary according to different embodiments. For example, while certain components or sub-components can be depicted as being distinct or separate from one another, it should be recognized that this distinction can be a logical distinction rather than a physical or actual distinction. Any or all of the components and sub-components can be combined with one another to perform the functions described herein, and any aspect or feature that is described as being performed by one component or sub-component can be performed by any or all of the other components and sub-components. Likewise, although the figures can depict a specific number of each component (e.g., a single controller, a single communication component, a single mobile device, etc.), this is not intended to be limiting and the system can include any number of each such component.

It should also be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments can be recited in or added to the claims, as well as negative limitations that define the inventive scope of the claims by features, functions, steps, or elements that are not within that scope.

It is to be understood that this disclosure is not limited to the particular embodiments described herein, as such can, of course, vary. It is also to be understood that the terminology used herein is for the purpose of describing particular embodiments only and is not intended to be limiting.

As used herein and in the appended claims, the singular forms “a,” “an,” and “the” include plural referents unless the context clearly dictates otherwise.

In general, terms such as “coupled to,” and “configured for coupling to,” and “secure to,” and “configured for securing to” and “in communication with” (for example, a first component is “coupled to” or “is configured for coupling to” or is “configured for securing to” or is “in communication with” a second component) are used herein to indicate a structural, functional, mechanical, electrical, signal, optical, magnetic, electromagnetic, ionic or fluidic relationship between two or more components or elements. As such, the fact that one component is said to be in communication with a second component is not intended to exclude the possibility that additional components can be present between, and/or operatively associated or engaged with, the first and second components.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities can optionally be present other than the entities specifically identified by the “and/or” clause, whether related or unrelated to those entities specifically identified. Thus, as a non-limiting example, a reference to “A and/or B”, when used in conjunction with open-ended language such as “comprising” can refer, in one embodiment, to A only (optionally including entities other than B); in another embodiment, to B only (optionally including entities other than A); in yet another embodiment, to both A and B (optionally including other entities). These entities can refer to elements, actions, structures, steps, operations, values, and the like.

What is claimed is:

1. A patient monitoring system, the system comprising:
a plurality of health sensors configured to measure one or more vital signs of a patient; and
a health application residing on the mobile device, the health application is configured to communicate with the plurality of health sensors and to receive data of the one or more vital signs measured by the plurality of health sensors.

2. The patient monitoring system of claim 1, wherein the plurality of health sensors comprises two or more of a blood oxygen level monitor, a blood pressure monitor, a thermometer, a hear rate monitor, and a breathing monitor.

3. The patient monitoring system of claim 1, wherein the plurality of health sensors is configured to communicate with the health application via Bluetooth.

4. The patient monitoring system of claim 1, wherein the health application is configured to display data of the one or more vital signs measured by the plurality of health sensors.

5. The patient monitoring system of claim 1, wherein the health application is configured to determine a location of the patient; and determine nearby pharmacies based on the location of the patient.

6. The patient monitoring system of claim 1, wherein the health application is configured to evaluate the data of the one or more vital signs to provide work clearance or to recommend further treatments.
